(19) United States
(12) Patent Application Publication
(10) Pub. No.: US 2009/0061027 A1
(43) Pub. Date: Mar. 5, 2009

(54) Composition For The Prevention and Treatment Of Common Cold Diseases

(76) Inventor: Georgios Pandalis, Glandorf (DE)

Correspondence Address: KILYK & BOWERSOX, P.L.L.C., 400 HOLIDAY COURT, SUITE 102, WARRENTON, VA, 20186, US

(21) Appl. No.: 12/281,850

(22) PCT Filed: Mar. 2, 2007
(86) PCT No.: PCT/EP2007/001829

§ 371 Date: Oct. 10, 2008

Related U.S. Application Data
(60) Provisional application No. 60/785,602, filed on Mar. 24, 2006.

(30) Foreign Application Priority Data
Mar. 24, 2006 (EP) 06006149.6

(51) Int. Cl. A61K 36/185; A61P 31/16

(52) U.S. Cl. 424/725

(57) ABSTRACT

The invention relates to the use of Cistus for producing a composition for the prophylaxis and/or treatment of common cold diseases. In particular, the composition can be used for the prophylaxis and/or treatment of the symptoms of a head cold.

The invention under consideration relates to the use of Cistus for the production of a composition or preparation for the prophylaxis and/or treatment of common cold diseases (viral colds).

Included among the typical common cold diseases are respiratory tract infections, such as head colds and inflammations of the tonsils and pharynx, as well as coughs and bronchitis. Usually these occur one after the other, but the cold can also remain restricted to the nose, throat or bronchia. Common cold diseases of this kind are also called “viral colds”. These are not to be confused with the influenza produced by influenza viruses, which shows a considerably longer and more serious progress of the disease and is, as a rule, associated with fever.

The common cold diseases mentioned are also caused by viruses. Because there are, for example, more than a hundred different types of viruses that can cause a head cold, it will scarcely be possible to develop a vaccine against it. Treatment of head colds or colds in general is therefore targeted at relieving the symptoms. Usually well-tried household remedies are used in these cases. For example, a very congested nose can be helped by the inhalation of hot steam. It allows the swelling in the nasal mucosa to go down and promotes the discharge of the mucous. This can be aided, for example, by the addition of a few drops of tea-tree oil or camomile oil into the hot water. It is also known that routine rinsing of the nose with a saline solution can reduce the susceptibility to head colds.

In addition to the self-help measures, medicines can help to constrict the vessels in the swollen nasal mucosa, leading to a soothing of the nasal mucosa. Nose drops for reducing the swelling of the nasal mucosa should not be used longer than two or three days, however. After this time, it is possible that when the drops are discontinued, the nasal mucosa will swell up all the more, and “rebound swelling” (rhinitis medicamentosa) develops.

Unlike chemical synthetic nasal sprays, phytopharmaceuticals have few side-effects. Even if used over a longer time period, they do not damage the nasal mucosa and do not lead to rhinitis medicamentosa. The sooner phytopharmaceuticals are applied, the more effective they are. They can already be used for support at the first signs of a cold. They also counteract the spread of infection.

For example, often echinacea preparations are taken for common cold diseases, whereby a great number of various medicines in variable phytochemical compositions are on the market. Controlled studies on the efficacy of these phytotherapeutic agents exist only to a limited degree, however, and with contradictory results. Just recently, however, a new study revealed that echinacea does not have the postulated efficacy. The study was conducted with three echinacea preparations with various phytochemical profiles, whereby these preparations were acquired by the extraction of E.-angustifolia roots with carbon dioxide, 60% ethanol or 20% ethanol. The total of 437 volunteers with rhinovirus infections who took part in this study received the medicine either as a prophylaxis seven days before exposure to the virus or for treatment at the time of the exposure. The study included a control group that received placebos. There was no significant difference between the three echinacea extracts and the placebo with regard to infection rate, severity of the symptoms, volume of the nasal secretion, leukocyte level, interleukin-8 concentration in the nasal douche water or quantitative virus titres (Deutsches Ärzteblatt 102, Issue 48 from 2 Dec. 2005, page A-3341/B-2822/C-2640 and the New England Journal of Medicine, 2005, 353, 341-348).

A further medicine on a botanical basis is an extract from the roots of Pelargonium reniforme or sidoides, which is marketed under the name Umckaloabo®. Umckaloabo® is traditionally used not only for respiratory tract illnesses, but also for gastrointestinal illnesses. The ingredients determining the efficacy are currently considered to be a number of antibacterial and immunomodulating components, such as coumarins and tannins. It is postulated that the extract develops antibacterial, antiviral and secretolytic effects, whereby the medicine should not be used by pregnant or nursing women, or by patients with liver or kidney diseases or an increased bleeding tendency, because it has not yet been possible to collect sufficient experience in this area. Moreover, Umckaloabo® is quite costly compared to other phytopharmaceuticals.

The object of the present invention is therefore to provide an antiviral composition for the prophylaxis and/or treatment of common cold diseases (viral colds), whereby the composition can be economically produced and causes no side-effects at all or only minor side-effects when administered.

This object is solved by the use of Cistus for producing a composition for the prophylaxis and/or treatment of common cold diseases (viral colds).

### DESCRIPTION OF THE FIRGURES

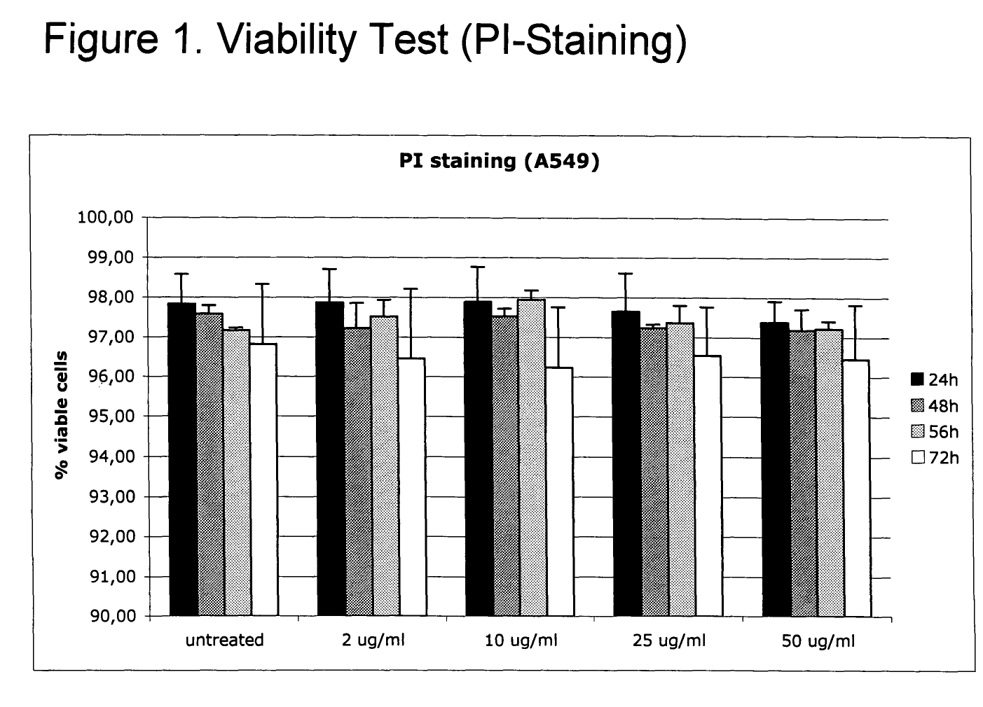

FIG. 1 shows the results of a viability test with propidium iodide staining of A549 cells that have been treated with Cistus extract.

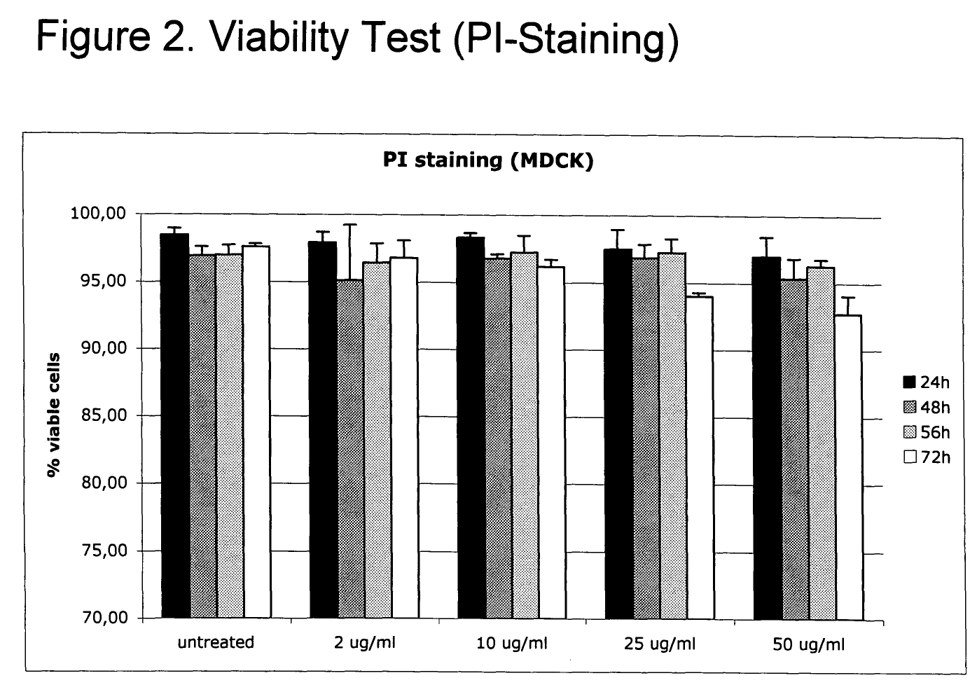

FIG. 2 shows the results of a viability test with propidium iodide staining of MDCK cells that have been treated with Cistus extract.

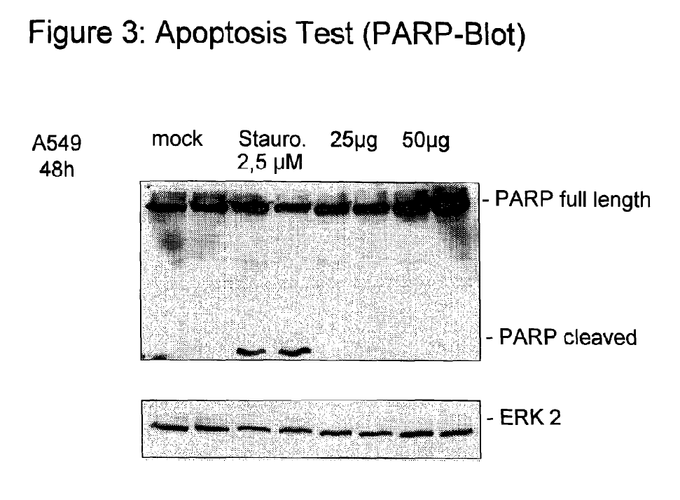

FIG. 3 shows the results of an apoptosis test of A549 cells that have been treated with Cistus extract.

### DETAILED DESCRIPTION OF THE INVENTION

Understood as common cold diseases for the purposes of the invention are inflammations of the respiratory tract, meaning, as a rule, the nose, pharynx, larynx, trachea and bronchia. The terms “common cold diseases” and “viral colds” are used synonymously in this case. A viral cold is distinguished from influenza in that the latter is caused only by influenza viruses.

A viral cold, on the other hand, is usually brought about by adenoviruses, coronaviruses and/or rhinoviruses.

Adenoviruses (Adenoviridae) belong to the family of the non-enveloped cubic DNA viruses and have a diameter of 60 to 90 nm. The genome consists of a linear double-stranded DNA approximately 36 kb long. One differentiates approximately 50 immunologically distinct types of adenoviruses, with approximately 35 human pathogenic types in the sub-genera A-F. The Adenoviridae family is divided into the genera Mastadenoviruses, which can infect mammals, and Aviadenoviruses, which are endemic in various bird species. Adenoviruses are characterised by unusual stability in the face of chemical and physical effects and tolerate the most adverse pH levels, which allows them a comparatively long survival time outside the host body.

Adenoviruses primarily cause illnesses of the respiratory tract. Depending on the particular serotype, however, a number of other illnesses can also be caused, for example, gastroenteritis, conjunctivitis, cystitis, pharyngitis or diarrhoeas. The symptoms of the respiratory tract illness caused by adenoviruses range from the common cold to bronchitis to pneumonia. In the case of patients with weakened immune systems, there is special susceptibility to serious complications from the adenovirus infections, such as ARDS (Acute Respiratory Distress Syndrome), for example. Furthermore, it is suspected that there is a correlation between the virus type Ad-36 and obesity in humans.

Coronaviruses, which belong to the genus Coronaviridae, generally cause mild illnesses of the upper respiratory tract in humans, seldom gastroenteritis and the Serious Acute Respiratory Syndrome (SARS), caused by the SARS-associated coronavirus SARS-CoV.

The coronaviruses are classified in the family of the enveloped pleomorphic RNA viruses and have a diameter of 70 to 160 nm. They have a single-stranded positive-sense RNA with a length of from 20 to 30 kb. The Coronaviridae genus is divided into three genera: the coronaviruses, the arteriviruses and the toroviruses. Of these, only the coronaviruses comprise human pathogenic viruses. The transmission of the viruses takes place through droplet infection (aerogenic), as a dirt or smear infection (faecal-oral) or even through simple contact (mechanical) with an infected person. Younger infected organisms in this case can become more seriously ill than older ones. Coronaviruses cause between 15 to 30% of the common cold diseases in humans with a slight fever, head cold, cough and sore throat.

An acute or chronic irritation of the nasal mucosa with the symptoms of itching, sneezing, secretion and congestion caused by infectious, allergic and non-allergic mechanisms is called rhinitis, nasal catarrh, coryza or colloquially, head cold. The pathogen is usually a genus of the picomavirus—the rhinovirus. The infection with rhinoviruses takes place through direct transmission, e.g., via contaminated hands or also via droplet infection.

More than 115 serotypes of this genus have been identified until now. Rhinoviruses have a single-stranded, positive-sense RNA (messenger RNA) with a length of from 7.2 to 8.5 kb. These are naked viruses with an icosahedral structure and a diameter of from 24 to 30 nm. The 10 to 15 nm-thick protein envelope (capsid) surrounding the RNA consists of 60 symmetrically arranged subunits, which are called protomers. Each protomer consists of the four capsid proteins VP1, VP2, VP3 and VP4. The multiple number of protomers is considered the cause of the antigenic versatility of the rhinoviruses.

As already mentioned, common cold diseases are usually caused by adenoviruses, coronaviruses and/or rhinoviruses. Depending on the type of infection virus, cold complaints such as head cold, coughing, hoarseness, sore throat, for example, caused by inflammation of the tonsils and pharynx, joint pain and head ache, chills, slight fever and exhaustion can occur. For the purposes of the invention, bronchitis and bronchial pneumonia are also counted as common cold diseases.

Of these common cold diseases, a head cold in the winter months occurs most frequently. It is caused by an infection with rhinoviruses, or, less frequently, with adenoviruses. The described composition or preparation is preferably used for the prophylaxis and/or treatment of head colds.

Furthermore, bacterial infections, which “set up” on the already existing virus infection, can occur with common cold diseases. Infections of this kind are called secondary bacterial infections or bacterial superinfections. Use of the composition according to the invention also concerns the prophylaxis and/or treatment of these secondary bacterial infections.

According to the invention, the Cistus plant is used for producing a composition for the prophylaxis and/or treatment of viral cold infections. 20 species are known in the Cistus genus:

C. albidus L.

C. chinamadensis Banares & P. Romero

C. clusii Dunal

C. cispus L.

C. heterophyllus Desf.

C. incanus (also called C. creticus)

C. inflatus Pourr. Ex Demoly (also called C. hirsutus Lam. or C. psilosepalus Sweet)

C. ladanifer L.

C. laurifolius L.

C. libanotis L.

C. monspeliensis L.

C. munbyi Pomel

C. ochreatus Chr. Sm. ex Buch

C. osbeckiifolius Webb ex Christ.

C. parviflorus Lam.

C. populifolius L.

C. pouzolzii Deute

C. salviifolius L.

C. sintenisii Litard. (also called C. albanicus E. F. Warburg ex Heywood)

C. symphytifolius Lam.

Preferably, the composition is produced from the species C. incanus. C. incanus includes two subspecies: C. incanus ssp. tauricus and C. incanus ssp. undulatus. Of these, the subspecies C. incanus ssp. tauricus is particularly preferably used for the composition.

The separate components of the Cistus plant are not yet known in full. It has already been shown, however, that Cistus has a high content of polyphenols. Of these polyphenols, the flavonoids are especially strongly represented.

Flavonoids basically consist of two aromatic and one oxygenated heterocyclic ring. Using structural differences on the O-heterocyclic ring, the flavonoids can be divided into the following six groups: flavonols, flavanols, flavanones, flavones, anthocyanins and isoflavanoids. Some of the components detected in Cistus plants are flavonols, such as quercetin and myricetin and their glycosides, flavan-3-ols (catechins), such as (+)-gallocatechin and (+)-gallocatechin-3-O-gallate, as well as dimers and oligomers of the catechins, the proanthocyanidins.

Further components are α-pinen, camphene and terpenoids, such as labda-7,13-dien-15-ol, 8α, 15-labdan diol, labdanolic acid, laurifolic acid, acetyl laurifolic acid, 8,13-epoxy-15-dimethoxy-ent-labdan, 3a-hydroxy-ent-13-epimanoyl oxide (=ribenol), salmanti acid, salmanti diol, halimic acid, dihydro halimic acid, 2β-hydroxy-dihydro halimic acid, cistodioic acid, cistodiol, ent-kauran-16,17-diol, dihydro-abietic-acid-methylester, cabraleone and 20,25-epoxy-24-hydroxy dammaran-3-on [R. Hegnauer, Chemotaxonomie der Pflanzen Vol. VIII S. 1989, 246-247].

The composition used according to the invention is produced from the aboveground parts of the plants. Preferably, the aboveground shoots of the plant that grow back in the same year are used. All elements of the aboveground part of the plant, such as leaves, stalks or blossoms, can be used. Preferably, the stalks are used with leaves and blossoms. The parts of the plant can be either dried or pressed out directly after the harvest, meaning in the raw state, after being broken up, where appropriate, in order to produce a juice from the pressing. In a further embodiment, the plant parts, in the raw state, are submitted to an extraction with a solvent, such as a maceration or percolation, for example. Alternatively, the plant parts can also be dried and/or subsequently broken into small pieces in a suitable manner before the extraction, by means of rubbing or cutting them, for example.

For the composition used according to the invention, dried and, where applicable, plant elements broken into small pieces, the pressed plant juice or an extract are used. According to the invention, the term “extract” is used representatively for all products that are obtained by means of an extraction with a solvent, such as with maceration or percolation.

Preferably, the composition is in the form of an extract from the Cistus plant.

Generally, an extraction with a suitable solvent takes place. Suitable solvents are water, alcohols, such as methanol, ethanol or isopropyl alcohol, or chlorinated solvents, such as dichloromethane, as well as acetone, acetylacetone, ethylacetate, ammonia or glacial acetic acid, but also supercritical carbon dioxide. Mixtures of the solvents mentioned can also be used. Preferably, water or a mixture of water with methanol or ethanol is used.

The extraction is normally carried out at temperatures of 25° C. to, where applicable, as high as the boiling point of the solvent used. Preferred is an extraction at 95 to 100° C.

Furthermore, fats, such as pork fat, waxes, such as beeswax, or oils, such as olive oil and almond oil, can be used for the extraction. Preferably, almond oil is used.

In order to achieve the highest possible yield, the plant material can be extracted a number of time. In this case, it is also possible to use different solvents in the various extraction steps or an extraction with a solvent can be followed by an extraction with a fat, wax or oil, or vice versa.

Through the extraction, a liquid, semi-solid or solid raw product is obtained, which can be used in this form for producing a composition for the prophylaxis and/or treatment of common cold diseases.

A maceration procedure is normally performed for five to nine days, preferably for seven days, at room temperature with a mixture of water and ethanol, by pouring the solvent mixture over the plant elements and letting this stand for the period of time mentioned.

According to the invention, a percolation of the plant parts is normally achieved by treating the parts with water at 95 to 100° C. for four to five hours by conducting the water through the plant parts.

The raw product obtained from an extraction with a solvent, such as a maceration or percolation, can also be concentrated and I or dried and/or further processed before use. The further processing can, for example, include cleaning steps known to the person skilled in the art, such as centrifugation, filtration and decanting, in order to remove suspended materials from the extract.

An extract obtained in this way can subsequently be further processed into a dry extract. To produce the dry extract, the solvent can be withdrawn from the liquid raw extract, the concentrated extract or the cleaned extract by, for example, spray drying, freeze drying or vacuum drying.

The composition from the Cistus plant can be used for the prophylaxis and/or treatment of common cold diseases in each of the forms described above.

The composition is preferably used for the prophylaxis and/or treatment of common cold diseases that are caused by rhinoviruses, adenoviruses or coronaviruses.

The composition according to the invention can therefore be administered as a medicine.

In addition to therapeutic use, the composition is also suitable for non-therapeutic prophylaxis and/or treatment of common cold diseases.

The composition can be applied in each of the application forms familiar to the person skilled in the art for both medical and non-medical use, e.g., as tablets, coated tablets, effervescent tablets, capsules, powders, granulates, sugar-coated tablets, ointments, creams, gels, solutions or sprays.

In galenic and other application forms, the composition can be processed with the customary galenic aids, such as tablet bonders, filling agents, preservative agents, tablet-opening agents, flow regulation agents, softening agents, wetting agents, dispersing agents, emulsifying agents, solvents, retarding agents, anti-oxidative agents, consistency regulators, penetration improvers and/or propellant gases.

Further elements, such as vitamins and minerals, can be added to the composition used according to the invention.

The composition can, for example, also be added to animal feed or foodstuffs, such as drinks. In the form of an extract, the composition itself can also be infused as tea. It is also possible, however, for hot water to be poured directly over the plant parts, for example, the leaves of the Cistus plants, for tea preparation. Furthermore, the composition can be a constituent of food supplements, whose ingestion in the winter months can contribute to strengthening the body's defences and consequently to preventing a head cold infection, for example.

In a further embodiment, the composition can be used according to the invention as a solution, in particular, a gargling solution, for the prophylaxis and/or treatment of common cold diseases, in particular of inflammations in the mouth and pharynx.

The composition can also be used mixed with constituents of other plants, in which case the constituents are preferably in the form of plant extracts. Preferably constituents of plants or plant extracts with a similar or synergetic effect are used.

The concentration of the composition in the application form varies, depending on the type of application. As a rule, the quantity of the composition amounts to between 0.5 and 1,000 mg per dosing unit for solid application forms. Preferably the quantity of the composition amounts to between 1 and 500 mg per unit. In liquid application forms, the composition can be in a concentration of 1 μg/ml to 100 mg/ml, preferably from 25 μg/ml to 50 mg/ml. In the case of semi-solid application forms, the content of the composition amounts to 1 to 90% by weight, preferably 5 to 75% by weight.

Preferably, the composition is administered in the form of a tablet. It is preferable for the composition be in the form of an extract in this case. Most especially preferred, the composition is in the form of a dry extract.

It is furthermore preferable for the composition to be administered in the form of emulsions, ointments, gels or creams for topical application. In this case, the composition is preferably used in the form of an extract in which the active substances are withdrawn from the plant by means of extraction with a fat, wax or oil. It is furthermore preferred for this extract to be further processed into a dry extract, which is subsequently mixed with or dissolved in a fat, wax or oil.

Furthermore, it is preferred for the composition to be in the form of an aerosol or room spray. Preferably a liquid or solid extract of Cistus is used for this. In addition to the extract, the aerosol or room spray can also contain pharmaceutically harmless substances, carrier media and auxiliary agents. The aerosol or room spay can be used for disinfecting objects and rooms with which cold viruses come into contact or could potentially come into contact, particularly means of transport of all types in which people, animals and/or foodstuffs are transported. For example, an airplane can be sprayed with the aerosol according to the invention or with the room spray according to the invention before takeoff, in order to prevent the spread of the cold viruses and consequently to minimise the risk of infection for people. The aerosol or room spray can also be sprayed in the presence of people, e.g., in waiting rooms, because it does not cause any toxic effects whatsoever in people.

Furthermore, the composition can also be administered as a nasal agent or as an inhalation solution. The nasal agent can be used as a nasal spray or as a nasal gel. For administration, various applicators and dispersion systems can be used.

The use of Cistus according to the invention is not restricted to people, but instead is also possible for animals, particularly mammals, such as pets or livestock.

The following example explains the invention under consideration.

A Cistus extract was tested with respect to its cell toxicity and cell viability, as well as its antiviral activity against rhinoviruses. To this end, the extract was dissolved in PBS (sterile) while being heated (1 h/100° C.) (stock solution 1 mg/ml). The dosage for the in vitro studies was 100 μg/ml system.

Human rhinovirus type 14 served as the virus isolate.

HeLa cells (human cervix carcinoma cell line) served as the host cell lines.

To determine the characteristics of the extract, the following examination methods were used.

### Microscopic Examinations

In the microscopic examinations, A549 lung epithelial cells and MDCK (Madin-Darby Canine Kidney cells) canine kidney epithelial cells were treated with various concentrations of the extract (2, 10, 25, 50 μg/ml) for different lengths of time (9 h, 24 h, 32 h, 48 h) and subsequently examined by light microscope. The experiments were checked twice.

### Viability Tests

In the viability tests, A549 lung epithelial cells and MDCK canine kidney epithelial cells were treated with various concentrations of the extract (2, 10, 25, 50 μg/ml) for different lengths of time (24 h, 48 h, 56 h, 72 h) and subsequently stained with propidium iodide in order to determine the relationship of dead and live cells by using flow cytometric methods. The experiments were conducted a total of four times.

### Examination of the Apoptotic Caspase Activation

To examine the apoptotic caspase activation, A549 cells were treated with 25 and 50 μg/ml of the extract for 48 hours. The cells were then lysed, and the cellular proteins were separated using gel electrophoresis and examined in the Western Blot with an antiPARP antibody (Poly(ADP-Ribose) polymerase, caspase substrate) for the apoptotic cleaving of this protein by caspases. The apoptosis-inductor staurosporine served as the positive control stimulus. The experiments were conducted in two parallel batches.

### EXAMPLE

Production of an Extract from Cistus incanus ssp. tauricus

The regrown aboveground shoots (leaves, blossoms and stalks) are used for extraction. The plant material is dried at room temperature outdoors in the shade, down to a residual water content of a maximum of 10%. Subsequently, the plant parts are cut to a size of ≦8 mm.

The cut plant parts are submitted to percolation at 95 to 100° C. with ten times the quantity of purified water Ph.Eur. for 4 to 5 hours. The solution produced is concentrated to 18 to 19% of the original volume by means of a plate evaporator at a steam temperature of 75 to 80° C. The content of the dry substance amounts to approximately 45%.

Using an evaporator with agitator, the content of the dry substance is increased to 50 to 51% by means of heating the extract for four hours at 110 to 114° C. at a reduced pressure (0.6 bar). The extract is subsequently boiled at 100.3° C. for 1 hour in order to obtain a dry substance of approximately 53%.

Finally, vacuum belt drying at 16 mbar with descending temperature gradients (140° C., 120° C., 90° C., 20° C.) is carried out. The content of the dry substance amounts to 92 to 93%. The extract is subsequently ground. The stock solution described in the preceding is then produced from this extract.

### Microscopic Examinations

In the microscope images of the examination series with MDCK cells and A549 cells, no significant changes with regard to the number of cells and the cell morphology in comparison to the untreated control samples could be detected in any of the extract concentrations used and the examined time values.

### Viability Test

Results of the viability tests are shown in FIG. 1 and 2, in which for comparison, the number of living cells from each of the samples is shown in summary as an average value of the four determinations made. No negative influence of the extract on the survival of MDCK or A549 cells could be detected in the result throughout the entire observation time of 72 hours.

### Examination of the Apoptotic Caspase Activation

The results of the examination of the apoptotic caspase activation are shown in FIG. 3, in which results of the Western Blot analysis for determining the caspase activity are shown. While the control stimulus staurosporine (Stauro) leads to an efficient cleaving of the caspase substrate poly (ADP-Ribose) polymerase (PARP cleaved lane), such activity cannot be shown in either the untreated (mock) or extract-treated cells (25 μg/ml, 50 μg/ml). A control blot against the protein ERK2 served as the control for uniform protein loading. In the result, it should be concluded that treatment with plant extract in the concentrations used and in the observation period did not lead to caspase activation and apoptotic induction.

The examinations on morphology, viability and caspase activation of the cells treated with Cistus extract show that concentrations of up to 50 μg/ml of the extract do not show any significantly toxic effect on the A549 and MDCK host cells used here, and induce neither increased necrotic nor apoptotic cell death. Furthermore, no significant reduction in the number of cells could be detected, so that the cell growth is also not restricted due to the extract effect.

### Examinations of Antiviral Activity

### Rhinoviruses Culture

Four T175 flasks with 80% confluent HeLa cells were inoculated with human rhinovirus type 14 (HRV) and incubated at 33° C. for one week. To do this, 50 μl HRV14 (virus titre 10⁸/ml TCID (Tissue Culture Infectious Dose)) in 16 ml infection medium (DMEM (Dulbecco's Modified Eagle Medium), 2% FCS (Foetal Calf Serum), 10-20 mM MgCl₂) are mixed and 4 ml added to each T175. Each T175 is then filled up with 10 ml infection medium, so that 14 ml infection medium is in each T175. As soon as 70% of the adherent cells dissolve, the virus is harvested.

### Purification of the Rhinoviruses

The virus supernatant is first centrifuged for 30 minutes at 3,000 rpm in order to remove the cell pellet. In the ultracentrifuge, the virus supernatant is centrifuged at 35,000 rpm (rotor type SW41 Ti in Beckman polyallomer tubes) at 4° C. for three hours on sucrose cushions (1.5 ml sucrose 65% in water, 300 μl 10×PBS (Phosphate-Buffered Saline), 1.2 ml water) and the pellet is incorporated into 100 μl infection medium. Further virus concentration takes place with a 100 kDa cut-off filter (Centricon YM100) at 3,300 rpm for one hour at 4° C. The retentate contains the purified virus concentrate; the filtrate is discarded.

### Examination of the Antiviral Activity (Tissue Culture Infectious Dose (TCID) Assay))

On the previous day, 5×104 HeLa cells are sown in DMEM medium per 96-well plate, so that the cells are approximately 70% confluent the next day. The DMEM medium is suctioned off the next day and replaced with 90 μl infection medium (DMEM, 2% FCS, 10-20 mM MgCl₂). The infection takes place in the highest concentration of 100 μg/ml with 10 μl of the virus-containing solution in the first 96-well row. After these 100 μl have been mixed by means of up and down pippetting, 10 μl of each is given to the second 96-well row. The procedure is repeated with further rows (1:10 dilution series). Two or three batches are conducted at the same time and used for the evaluation.

The plate processed in this way is incubated in the incubator for five days at 33° C. On the fifth day, the plate is washed at least twice with PBS and stained with 100 μl crystal violet per 96-well (0.07% in pure ethanol) for five hours. The plate is subsequently washed in water and knocked a number of times (approximately 10 times) and dried. A blue colouration indicates living cells. Dead cells were washed out of the wells and leave behind a white background.

In order to test the effect of the Cistus extract on the infectivity of HRV14, either 100 μg/ml Cistus extract was added to the infection medium or the viruses were additionally pre-treated with 100 μg/ml Cistus extract for one hour.

In the result, it could be seen that the Cistus extract was able to prevent the infection and therefore the destruction of the cell layer in both the infection medium and after additional pre-incubation of the viruses in all of the batches conducted. In the concentration used, the Cistus extract had no detectable damaging influences on the cells whatsoever. This proves an inhibitory effect of the Cistus extracts on the infectivity of rhinoviruses.

1. A method for preventing or treating a common cold disease comprising administering a composition comprising Cistus to a patient.

2. The method according to claim 1, wherein the common cold disease comprises a primary infection, caused by rhinoviruses, adenoviruses or coronaviruses.

3. The method according to claim 1 wherein said common cold disease is a head cold.

4. The method according to claim 1, wherein said Cistus is from a plant Cistus incanus.

5. The method according to claim 4, wherein the aboveground parts of the plant are used.

6. The method according to claim 1, wherein the composition is in liquid, dry or semi-solid form.

7. The method according to claim 1, wherein the composition is used in the form of an extract.

8. The method according to claim 7, wherein the extract is an aqueous extract or an alcoholic extract.

9. The method according to claim 1, wherein the composition is administered orally or topically.

10. The method according to claim 1, wherein the composition is present as a nasal agent or inhalation mixture.

11. The method according to claim 1, wherein the composition is present as an aerosol or room spray.

12. The method according to claim 1, wherein the composition is present in the form of a tablet, coated tablet, effervescent tablet, capsule, powder, granulate or sugar-coated tablet.

13. The method according to claim 1, wherein the composition is present in the form of an ointment, gel or cream.

14. The method according to claim 1, wherein the composition is present as a gargling solution or plant juice.
